(19) United States
(12) Patent Application Publication
(10) Pub. No.: US 2003/0180814 A1
(43) Pub. Date: Sep. 25, 2003

(54) Direct immunosensor assay

(76) Inventors: Alastair Hodges, Blackburn South (AU); Ronald Chatelier, Bayswater (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/105,050

(22) Filed: Mar. 21, 2002

(51) Int. Cl. G01N 33/53; G01N 33/542; C12Q 1/54

(52) U.S. Cl. 435/7.9; 435/14

(57) ABSTRACT

This invention describes a quantitative, inexpensive, disposable immunosensor that requires no wash steps and thus generates no liquid waste. Moreover, in preferred embodiments of the sensor no timing steps are required of the user, and the sensor can be readily adapted to antigen-antibody interactions over a wide kinetic range.

### FIELD OF THE INVENTION

The present invention relates to a device and method for performing immunoassays. The device comprises a disposable immunosensor.

### BACKGROUND OF THE INVENTION

Biomedical sensors are used to report the presence and/or concentration of a wide variety of analytes. When the analyte is a protein, then the sensing element used is usually an antibody since the interaction of the antibody with the protein (antigen) is very specific. Such immunoassays usually fall into two categories: a “yes/no answer” obtained, e.g., by simple visual detection, or a concentration of the antigen determined by a quantitative method. Most of the quantitative methods involve expensive pieces of equipment such as scintillation counters (for monitoring radioactivity), spectrophotometers, spectrofluorimeters (see, e.g., U.S. Pat. No. 5,156,972), surface plasmon resonance instruments (see, e.g., U.S. Pat. No. 5,965,456), and the like. It would therefore be advantageous to develop a quantitative immunoassay that is both inexpensive and simple enough to use to be suitable for home or field use. Such an immunosensor requires no centrifugation, dilution, pipetting, washing, or timing steps, and generates minimal waste.

Conventional immunoassays are classified into two categories: competition assays and sandwich assays. In a competition assay, the antigen in the test sample is mixed with an antigen-probe complex (commonly referred to as a reporter complex) and the mixture then competes for binding to the antibody. The probe may be a radioisotope, an enzyme, a fluorophore, or a chromophore. In a sandwich immunoassay, the antigen in the test sample binds to the antibody and then a second antibody-probe complex binds to the antigen. In these prior art assay methods, one or more washing steps are usually required. The washing steps introduce complexity into the assay procedure and can generate biohazardous liquid waste. It would therefore be advantageous to develop a device for performing an immunoassay that does not require any washing steps and is suitable for a single use as a disposable device.

### SUMMARY OF THE INVENTION

A quantitative, inexpensive, disposable immunosensor that requires no wash steps and thus generates no liquid waste is provided. For immunosensors of certain embodiments, no timing steps are required of the user, and the sensor can be readily adapted to antigen-antibody interactions over a wide kinetic range. The sensors of the preferred embodiments have a number of potential advantages. Such sensors may be simpler to fabricate, as reagents may be deposited in a single step and/or on only one portion of the reaction chamber or a support contained therein.

The sensors may utilize a pseudo-antigen-probe complex, a modified-antigen-probe complex, or an antigen-probe complex. The term “pseudo-antigen,” as used herein, is a broad term and is used in its ordinary sense, including, without limitation, antigens other than the antigen of interest that bind to the immobilized antibody, but not as strongly as the antigen of interest. The term “modified-antigen,” as used herein, is a broad term and is used in its ordinary sense, including, without limitation, antigens that have been chemically or otherwise modified such that the modified-antigen binds to the immobilized antibody, but not as strongly as the antigen of interest. The antigen of the antigen-probe complex, which may be the same as or different than the antigen of interest, by virtue of being bound to a probe will bind to the immobilized antibody, but not as strongly as the antigen of interest, which is in an unbound state. While the preferred embodiments are discussed primarily in regard to a pseudo-antigen, it is understood that an antigen-probe complex or modified-antigen may be substituted for a pseudo-antigen.

It may be easier to ensure that the ratio of antibody to antigen-probe, modified-antigen-probe, or pseudo-antigen-probe in the reaction chamber is correct as this will essentially occur automatically when the antigen-probe, modified-antigen-probe, or pseudo-antigen-probe is bound to the antibody during manufacture of the sensor, in contrast to prior art methods where the correct ratio is typically achieved by controlling reagent lay-down rates and surface densities. The sensor of preferred embodiments may also be particularly well suited to slower immuno-reaction kinetics, wherein the binding processes may be slow. The use of a non-human pseudo-antigen in the manufacture of the sensor may reduce the likelihood of transmission of communicable diseases when the sensor contacts a drop of blood on the patient's finger.

In a first embodiment, a disposable device for use in detecting a target antigen in a fluid sample is provided, the device including a reaction chamber; an immobilized antibody fixed within the reaction chamber; a reporter complex including a probe and a reporter complex antigen, wherein the probe is linked to the reporter complex antigen, wherein the reporter complex antigen is bound to the immobilized antibody, and wherein the reporter complex antigen binds less strongly than the target antigen to the immobilized antibody; a detection chamber; a sample ingress to the reaction chamber; and a sample passageway between the reaction chamber and the detection chamber.

In an aspect of the first embodiment, the reporter complex antigen may be a target antigen, a pseudo-antigen, or a modified-antigen. The probe may include radioisotopes, chromophores, or fluorophores.

In an aspect of the first embodiment, the probe may include an enzyme, such as glucose dehydrogenase. When the probe is an enzyme, the detection chamber may further include an enzyme substrate, for example, an oxidizable substrate such as glucose. The detection chamber may also further include a mediator, such as dichlorophenolindophenol, or complexes between transition metals and nitrogen-containing heteroatomic species, or ferricyanide. The device may further include a buffer that adjusts the pH of the sample, such as a phosphate or a mellitate. The device may also include a stabilizer, wherein the stabilizer stabilizes one or more of the target antigen, the reporter complex antigen, the enzyme, and the immobilized antibody. The enzyme substrate may be supported on a detection chamber interior surface.

In an aspect of the first embodiment, the immobilized antibody may be supported on a reaction chamber interior surface.

In an aspect of the first embodiment, the device also includes a support material. The support material may be contained within the detection chamber, and may include a first substance such as an enzyme substrate, a mediator, or a buffer, that may be supported on or contained within the support material. The support material may be contained within the reaction chamber, and may include a second substance such as immobilized antibody, the reporter complex, or an agent that prevents non-specific binding of proteins to a reaction chamber internal surface, that may be supported on or contained within the support material. The support material may include a mesh material, for example a mesh material including a polymer such as polyolefin, polyester, nylon, cellulose, polystyrene, polycarbonate, polysulfone, or mixtures thereof. The support material may include a fibrous filling material, such as a fibrous filling material including a polymer such as polyolefin, polyester, nylon, cellulose, polystyrene, polycarbonate, polysulfone, or mixtures thereof. The support material may include a porous material, such as a sintered powder, or a macroporous membrane, for example, a macroporous membrane including polymeric material such as polysulfone, polyvinylidene difluoride, nylon, cellulose acetate, polymethacrylate, polyacrylate, or mixtures thereof. The support material may include a bead.

In an aspect of the first embodiment, the detection chamber includes a first electrode and a second electrode. At least one of the first electrode and the second electrode includes a material such as aluminum, copper, nickel, chromium, steel, stainless steel, palladium, platinum, gold, iridium, carbon, carbon mixed with binder, indium oxide, tin oxide, a conducting polymer, or mixtures thereof.

In an aspect of the first embodiment, a detection chamber wall may be transparent to a radiation emitted or absorbed by the probe, and the radiation is indicative of a presence or absence of the reporter complex in the detection chamber.

In an aspect of the first embodiment, the device includes a detector that detects a condition wherein the reaction chamber is substantially filled.

In an aspect of the first embodiment, the device includes a piercing means that forms a detection chamber vent in a distal end of the detection chamber. The device may also include a reaction chamber vent at a distal end of the reaction chamber.

In an aspect of the first embodiment, the target antigen includes a human C-reactive protein. The reporter complex antigen may include a monomeric C-reactive protein. Alternatively, the reporter complex antigen may include a C-reactive protein derived from a non-human species, or a chemically-modified C-reactive protein, wherein an affinity of the chemically-modified C-reactive protein to the antibody is less than an affinity of the human C-reactive protein to the antibody

In an aspect of the first embodiment, a wall of the detection chamber or a wall of the reaction chamber includes a material such as polyester, polystyrene, polycarbonate, polyolefin, polyethylene terephthalate, or mixtures thereof. The wall of the detection chamber or the wall of the reaction chamber may also include a filler, such as titanium dioxide, carbon, silica, glass, and mixtures thereof.

In an aspect of the first embodiment, the probe includes an enzyme cofactor, such as flavin mononucleotide, flavin adenine dinucleotide, nicotinamide adenine dinucleotide, or pyrroloquinoline quinone. The enzyme co-factor may be linked to the reporter complex antigen through a flexible spacer. The detection chamber may also include an enzyme substrate, or an apoenzyme.

In an aspect of the first embodiment, the probe includes an enzyme activity regulator, such as a kinase or phosphorylase. The detection chamber may also include an enzyme substrate, or an enzyme.

In an aspect of the first embodiment, the probe includes a protein subunit which is part of a multi-subunit enzyme.

In a second embodiment, a method for determining an amount of a target antigen in a fluid sample is provided, the method including the steps of: placing the fluid sample in a reaction chamber containing an immobilized antibody and a reporter complex including a probe linked to a reporter complex antigen, wherein the antibody is fixed within the reaction chamber, wherein the reporter complex antigen is bound to the immobilized antibody, and wherein the reporter complex antigen binds less strongly than the target antigen to the immobilized antibody; dissociating a portion of the reporter complex antigen from the immobilized antibody into the fluid sample; binding a portion of the target antigen to the immobilized antibody; transferring the fluid sample to a detection chamber; and determining an amount of reporter complex in the fluid sample, wherein the amount of reporter complex is indicative of the amount of target antigen initially in the fluid sample.

In an aspect of the second embodiment, the step of transferring the fluid sample to a detection chamber includes transferring the fluid sample to an electrochemical cell having a first electrode and a second electrode. The step of determining an amount of reporter complex in the fluid sample may also include: applying a potential between the first electrode and the second electrode in the electrochemical cell; and measuring a current, wherein the current is indicative of an amount of reporter complex present in the fluid sample, and wherein the amount of reporter complex is indicative of the amount of target antigen.

In an aspect of the second embodiment, the step of transferring the fluid sample to a detection chamber includes transferring the fluid sample to a detection chamber including an electromagnetic radiation transmissive portion. The step of determining an amount of reporter complex in the fluid sample may also include the steps of: exposing the electromagnetic radiation transmissive portion to electromagnetic radiation, whereby the electromagnetic radiation passes through the fluid sample or reflects from the fluid sample; and monitoring a property of the electromagnetic radiation after it passes through the fluid sample or reflects from the fluid sample, wherein the property is indicative of an amount of reporter complex present in the fluid sample, and wherein the amount of reporter complex is indicative of the amount of target antigen.

### BRIEF DESCRIPTION OF THE DRAWINGS

FIG. 1 shows a top view (not to scale) of an immunosensor of a first preferred embodiment that incorporates an electrochemical cell.

FIG. 2 shows a cross-sectional view (not to scale) along line A-A′ of an embodiment of the immunosensor of FIG. 1.

FIG. 3 shows a top view (not to scale) of an immunosensor of a preferred embodiment that incorporates an electrochemical cell.

FIG. 4 shows a cross-sectional view (not to scale) along line B-B′ of an embodiment of the immunosensor of FIG. 3.

### DETAILED DESCRIPTION OF THE PREFERRED EMBODIMENTS

The following description and examples illustrate a preferred embodiment of the present invention in detail. Those of skill in the art will recognize that there are numerous variations and modifications of this invention that are encompassed by its scope. Accordingly, the description of a preferred embodiment should not be deemed to limit the scope of the present invention.

A sensor strip is provided that contains two chambers: a reaction chamber and a detection chamber. A sample is received in the reaction chamber, wherein components of the sample undergo an immuno-reaction. One or more products of the immuno-reaction are detected in the detection chamber in order to quantitate the antigen present in the sample. The reaction chamber and detection chamber are arranged such that sample may flow from the reaction chamber into the detection chamber.

After the immuno-reaction has taken place in the reaction chamber, at least some of the reacted sample is transferred to the detection chamber, where the presence of a probe is detected and analyzed to obtain a result. It is preferred that sufficient sample is transferred such that the detection chamber is sufficiently filled, namely, that sufficient sample is transferred to the detection chamber such that the presence of a probe may be detected and analyzed by the detection method employed.

The reaction chamber contains antibodies to the antigen of interest immobilized within it. The antibodies can be immobilized on a wall of the chamber itself. Alternatively the antibodies may be immobilized on a support contained within the reaction chamber. Suitable supports include, but are not limited to, fibrous materials, macroporous materials, powdered materials, or, in particularly preferred embodiments, beads of a material such as are commonly known in the art for supporting antibodies.

In the preferred embodiments, the immobilized antibodies are bound to what is referred to as a “pseudo-antigen” linked to a probe. The pseudo-antigen-probe binds to the immobilized antibody, but not as strongly as the antigen of interest. If, for example, the antigen to be detected is a human protein, then a suitable pseudo-antigen-probe may include an animal version of the same protein, such as a dog protein or a pig protein, linked to the probe. In this example, antibodies to the human version of the protein are immobilized in the reaction chamber and the animal version of the protein, linked to a suitable probe, is bound to the immobilized antibody to form an antibody-pseudo-antigen-probe complex.

When sample fills the reaction chamber, a small fraction of the pseudo-antigen-probe dissociates into solution, since it is relatively weakly bound to the antibody. A dynamic equilibrium will exist between bound pseudo-antigen-probe and free pseudo-antigen-probe, leaving some free antibody binding sites. If there is antigen in the solution, then it will strongly bind to the free antibody binding sites in preference to the pseudo-antigen-probe and so leave the pseudo-antigen-probe in solution. This process will continue until substantially all of the antigen in the sample has bound to the antibodies and there is an equal amount of pseudo-antigen-probe free in the solution. Thus each antigen that binds to an immobilized antibody will displace one pseudo-antigen-probe into solution.

When all, or a pre-determined fraction, of the antigen in the sample is bound to the immobilized antibodies, the concentration of pseudo-antigen-probe in solution reflects the original concentration of antigen in the sample. In the preferred embodiments, the equilibrium between free and bound pseudo-antigen-probe is relied upon to ensure that antigen in solution ends up bound to the antibody in preference to the pseudo-antigen-probe. Hence, a pseudo-antigen-probe is employed that binds more weakly to the antibody than the target antigen, but there is no need to physically remove the pseudo-antigen-probe from the antibody prior to sample introduction, as in certain prior art methods.

After the immuno-reactions have taken place, the liquid sample containing any pseudo-antigen-probe liberated from the antibodies is transferred to the detection chamber. In the detection chamber, the concentration of pseudo-antigen-probe present in the sample is measured and a result obtained.

A small amount of the pseudo-antigen-probe may dissociate into solution even in the absence of antigen in the sample, as a result of the bound and free pseudo-antigen-probe reaching equilibrium in solution. If this occurs, then the signal generated in the detection chamber due to this free pseudo-antigen-probe is treated as a background signal, which is subtracted from the antigen concentration result as part of the analysis procedure.

In copending application Ser. No. 09/616,433 filed Jul. 14, 2000, incorporated herein by reference in its entirety, an immunoassay strip with a linked immuno-reaction and detection chamber is described. Unlike the sensor described herein, which employs a pseudo-antigen-probe initially complexed with an antibody immobilized on a surface within the reaction chamber, in the sensor of application Ser. No. 09/616,433, prior to the introduction of sample into the reaction chamber, antibodies are immobilized on one surface and antigen-probe is immobilized on another surface of the reaction chamber. When sample is introduced into the reaction chamber, the antigen-probe dissolves into the solution and competes with antigen in the sample for the antibody sites. The method of using the sensor of application Ser. No. 09/616,433 relies primarily on kinetic factors to ensure that the antigen binds to the antibody (by getting there first) in preference to the antigen-probe. Hence, there is a need to spatially remove the antigen-probe from the antibody in the reaction chamber, and the sensor can function when the antigen and the antigen-probe bind with equal strength to the antibody.

In preferred embodiments, the sensor is a single step, no-wash immunosensor. The sensor is a single use, disposable device that employs a reaction chamber and a detection chamber. Any suitable detection method can be utilized. Suitable detection methods include, for example, visual detection wherein the development of a color is observed, or spectroscopic detection wherein reflected or transmitted light is used to measure changes in light absorbance. In a preferred embodiment, the detection method is electrochemical, wherein the electrical current or potential related to the products of immuno-reactions is measured.

Methods and devices for obtaining electrochemical measurements of fluid samples are discussed further in copending U.S. patent application Ser. No. 09/616,556, filed on Jul. 14, 2000, which is incorporated herein by reference in its entirety.

The timing of the various test stages, i.e., the reaction stage and the detection stage, may be done manually. Alternatively, timing may be done automatically in response to a trigger signal generated when the reaction chamber and/or detection chamber is filled.

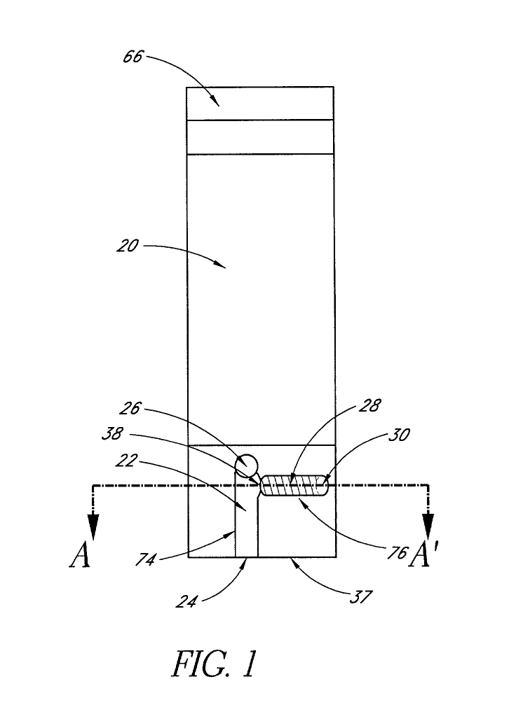

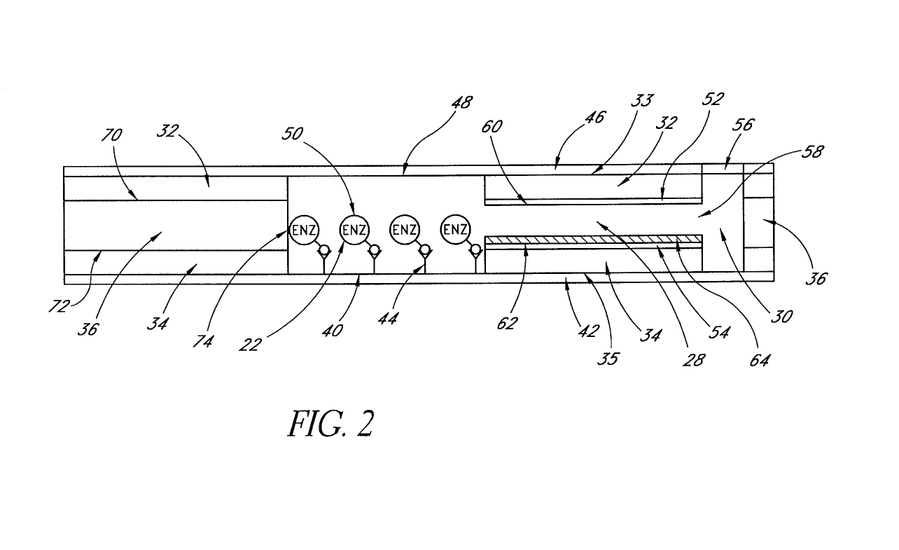

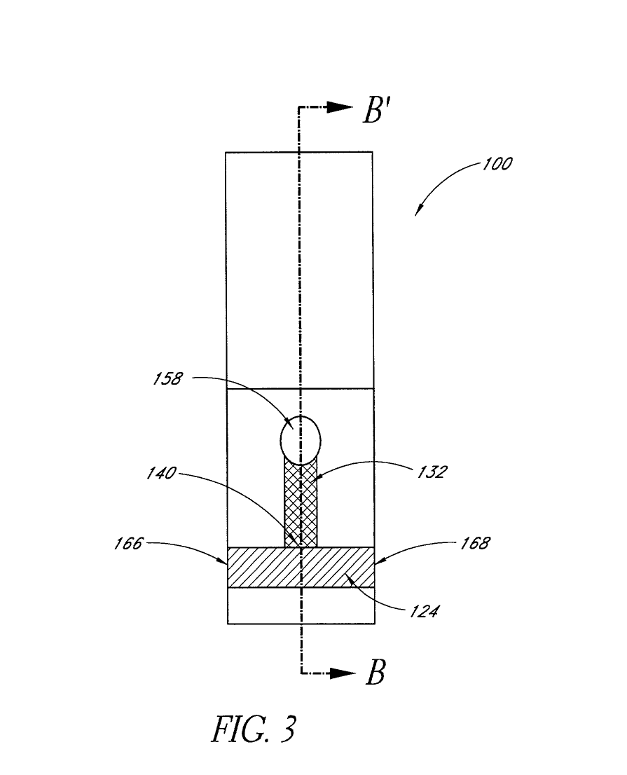

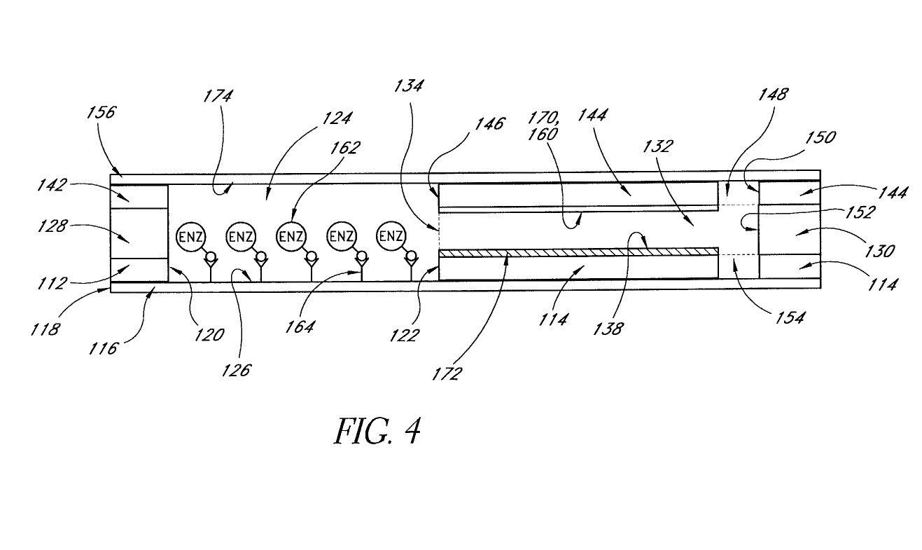

Embodiments of sensors suitable for use with electrochemical detection are illustrated in FIGS. 1 and 2 and in FIGS. 3 and 4. FIG. 1 is a top view of a first embodiment of a sensor strip and FIG. 2 is a cross-sectional view, showing details of the reaction chamber and the detection chamber. FIG. 3 is a top view of a second embodiment of a sensor strip and FIG. 4 is a cross-sectional view, showing details of the reaction chamber and the detection chamber.

The Sensor

The immunosensors of the present invention may be prepared using well-known thin layer device fabrication techniques as are used in preparing electrochemical glucose sensing devices (see, e.g., U.S. Pat. No. 5,942,102, incorporated herein by reference in its entirety). Such techniques, with certain modifications, may also used to prepare immunosensors utilizing non-electrochemical detection methods.

In the preferred embodiments of the immunosensors illustrated in FIGS. 1 and 2 and in FIGS. 3 and 4, the detection chamber comprises an electrochemical cell. The immunosensors may be prepared by assembling various thin layers of suitably shaped materials according to thin layer sensor fabrication methods as are well known in the art. Typically, the fabrication process involves sandwiching one or more spacer layers between a top layer and a bottom layer.

In a preferred embodiment, the sensor 20 is an electrochemical cell 28 utilizing an enzyme, e.g., glucose oxidase or glucose dehydrogenase, as the probe, as illustrated in FIG. 1, a top view of such a sensor 20, and FIG. 2, a cross section of the sensor through line A-A′. The reaction chamber 22 and detection chamber 28 are prepared by forming an aperture extending through a sheet of electrically resistive material 36. The aperture is shaped such that it defines a sidewall of both the reaction chamber 22 and the detection chamber 28, as well as the sample passageway 38 between the two chambers 22 and 28. By extending the aperture from the proximal end 24 of the reaction chamber 22 through to the edge of the sheet 37, the sample ingress 24 is also formed. In one embodiment, the thickness of the sheet 36 defines the entire height of the reaction chamber 22 and detection chamber 28, which are the same. In another embodiment, the height of the reaction chamber 22 is greater than that of the detection chamber 28. A reaction chamber 22 of greater height than the detection chamber 28 is prepared by layering multiple sheets 32, 34, and 36 together. The middle sheet 36 of the layer has an aperture defining the sidewalls 74 and 76 of both the reaction chamber 22 and detection chamber 28 as described above. This middle layer 36 is sandwiched between two or more additional layers 32 and 34, the additional layers 32 and 34 having an aperture defining the side wall 74 of the reaction chamber 22 only, the layers 32 and 34 thereby defining end walls 60 and 62 of the detection chamber 28. In this embodiment, the end walls 60 and 62 of the detection chamber comprise electrodes 52 and 54, which may be prepared as described below.

As illustrated in FIG. 2, antibodies 44 are tethered to the bottom 40 of the reaction chamber 22. The pseudo-antigen-probe 50 is complexed to the antibodies 44. The antibody may be tethered to any suitable surface within the reaction chamber, e.g. tethered to a wall or on a surface of a support within the reaction chamber 22.

A first thin electrode layer 52 is mounted or formed on one side 70 of the sheet of electrically resistive material 36, extending over the aperture forming the detection chamber 28 and forming an end wall 60. The layer 52 may be adhered to the sheet 36, e.g., by an adhesive. Suitable adhesives include, for example, heat activated adhesives, pressure sensitive adhesives, heat cured adhesives, chemically cured adhesives, hot melt adhesives, hot flow adhesives, and the like.

The electrode layer 52 may be prepared by coating (e.g., by sputter coating as disclosed in WO97/18464, by screen printing, or by any other suitable method) a sheet of electrically resistive material 32 with a suitable material, for example, aluminum, copper, nickel, chromium, steel, stainless steel, platinum, palladium, carbon, carbon mixed with a binder, indium oxide, tin oxide, mixed indium/tin oxides, gold, silver, iridium, mixtures thereof, conducting polymers such as polypyrrole or polyacetylene, and the like. If electrode 52 is to be used as a cathode in the electrochemical cell, then suitable materials include, for example, aluminum, copper, nickel, chromium, steel, stainless steel, platinum, palladium, carbon, carbon mixed with a binder, indium oxide, tin oxide, mixed indium/tin oxides, gold, silver, iridium, mixtures thereof, conducting polymers such as polypyrrole or polyacetylene, and the like. If electrode 52 is to be used as an anode in the electrochemical cell or is to come into contact with oxidizing substances during sensor manufacture or storage, then suitable materials include, for example, platinum, palladium, carbon, carbon mixed with a binder, indium oxide, tin oxide, mixed indium/tin oxides, gold, silver, iridium, mixtures thereof, conducting polymers such as polypyrrole or polyacetylene, and the like. Materials suitable for use as electrodes 52 and 54 are compatible with the reagents present in the sensor 20, namely, they do not react chemically with reagents at the potential of choice or during sensor fabrication and storage.

A second thin electrode layer 54 is mounted on the opposite side 72 of the electrically resistive material 36, also extending over the aperture forming the detection chamber 28, so as to form a second end wall 62. In this embodiment, the inert, electrically insulating layer 36 separates the electrode-bearing substrates 32 and 34. Preferably, insulating layer 36 keeps layers 32 and 34 at a predetermined separation. Provided this separation is small enough, e.g., less than or equal to about 500 microns, the current flowing between the electrodes 52 and 54 will be directly proportional to the concentration of reduced mediator after a suitably short time relative to the detection time employed. In this embodiment, the rate of current rise is directly related to the rate of the enzyme reaction and therefore the amount of enzyme present.

In certain embodiments, an electrode configuration other than an opposing relationship may be preferred, for example, a side-by-side relationship, or a parallel but offset relationship. The electrodes may be identical or substantially similar in size, or may be of different sizes and/or different shapes. The electrodes may comprise the same conductive material, or different materials. Other variations in electrode configuration, spacing, and construction or fabrication will be apparent to those of skill in the art.

In a preferred embodiment, the electrode layers 52 and 54 are mounted in a parallel opposing relationship at a distance of less than or equal to 500, 450, 400, 350, 300, 250, or 200 microns, and more preferably from about 5, 10, 15, 20, 25, 30, 35, 40, 45, or 50 microns to about 75, 100, 125, 150, or 175 microns. In certain embodiments, however, it may be preferred that the electrode spacing is greater than 500 microns, for example, 600, 700, 800, 900, or 1000 microns, or even greater than 1, 2, 3, 4, or 5 millimeters.

The volume of the detection chamber or the reaction chamber is typically about 0.3 microliters or less to about 100 microliters or more, preferably about 0.5, 0.6, 0.7, 0.8, or 0.9 microliters to about 20, 30, 40, 50, 60, 70, 80, or 90 microliters, and most preferably about 1.5, 2, 2.5, 3, 3.5, 4, 4.5, or 5 microliters to about 6, 7, 8, 9, 10, 12, 14, 16, or 18 microliters. However, in certain embodiments, larger or smaller volumes may be preferred for one or both of the reaction chamber and the detection chamber.

The electrodes 54 and 52 in the detection chamber 28 can be placed in electrical connection with a meter (not shown) through the connection end 66. The connectors (not shown) are in electrical connection with the electrodes 54 and 56 in the detection chamber 28 via conducting tracks (not shown). In the preferred embodiment illustrated in FIG. 1, these conducting tracks consist of extensions of the films of conductor 52 and 54 coated onto the internal surfaces of 32 and 34. The meter in connection with the connection area 66 is capable of applying a potential between the electrodes 52 and 54 in the detection chamber 28, analyzing the electrical signals generated, displaying a response, optionally storing the response in memory, and optionally transmitting stored responses to an external device such as a printer or computer.

In other embodiments utilizing electrochemical detection, stripes of conducting material on one or both internal faces of the detection chamber are typically used, with at least two electrodes present, namely, a sensing electrode and a counter/reference electrode. Optionally, a third electrode, serving as a separate reference electrode, may be present.

When utilizing potentiometric detection methods, the meter is capable of measuring the potential difference between a sensing electrode and a reference electrode, but need not be capable of applying a potential between the electrodes.

In embodiments wherein visual detection or reflectance spectroscopy is the detection method used, the layers 32 and 46 and/or layers 34 and 42 are transparent to the wavelength of radiation that is to be observed. In the case of visual detection, a simple color change in the detection chamber 28 is observed. In the case of reflectance spectroscopy, detection radiation is shone through layers 32 and 46 and/or layers 34 and 42, and radiation reflected from the solution in the detection chamber 28 is analyzed. In the case of transmission spectroscopy as the detection method, layers 32, 46, 34, and 42 are transparent to radiation at the wavelength of choice. Radiation is shone through the sample in the detection chamber 28 and the attenuation of the beam is measured.

In a preferred embodiment, layer 36 comprises a substrate with a layer of adhesive (not shown) coated on its upper surface 70 and lower surface 72. Examples of materials suitable for the substrate of layer 36 include polyester, polystyrene, polycarbonate, polyolefins, and, preferably, polyethylene terephthalate. These may be native materials or may be filled with suitable fillers to confer desirable optical or mechanical properties. Examples of materials suitable as fillers include, but are not limited to, titanium dioxide, carbon, silica, and glass. Examples of suitable adhesives are pressure sensitive adhesives, heat and chemically curing adhesives and hot melt and hot flow adhesives. Alternatively, the spacer layers themselves may consist of a suitable adhesive.

If a sample ingress 24 has not already been formed earlier in the fabrication process, then one is provided, for example, by forming a notch (not illustrated) in the edge 37 of the device 20 that intersects the reaction chamber 22.

The dashed circle in FIG. 1 denotes an aperture 30 piercing layers 32, 34, and 36 but not layers 42 and 46, the aperture in layer 34 opening into the detection chamber 28. Since layers 42 and 46 are not pierced initially, the only opening to the atmosphere of the detection chamber 28 is the sample passageway 38 opening into the reaction chamber 22. Thus, when the reaction chamber 22 fills with sample, the sample passageway 38 to the detection chamber 28 is blocked. This traps air in the detection chamber 28 and substantially prevents it from filling with sample. A small amount of sample will enter the detection chamber 28 during the time between when the sample first contacts the opening 38 to the detection chamber 28 and when the sample contacts the far side of the opening 38. However, once the sample has wet totally across the opening 38 to the detection chamber 28, no more filling of the detection chamber 28 will take place. The volume of the reaction chamber 22 is typically chosen so as to be at least equal to and preferably larger than the volume of the detection chamber 28. By opening the vent 30 to the atmosphere, sample is transferred to fill the detection chamber 28. The vent may be opened by means of a needle connected to a solenoid in the meter.

An immunosensor 100 of another embodiment, as depicted in FIGS. 3 and 4, may be prepared as follows. A first shaped layer 112 and a second shaped spacer 114 layer of similar thickness are each situated atop a bottom layer 116. The first spacer layer 112 is rectangular in shape, and is situated at the proximal edge 118 of the bottom layer 116. The second spacer layer 114 is also rectangular in shape, and is situated on the bottom layer 116 at a distance apart from the first spacer layer 112. The distal edge 120 of the first spacer layer 116 and the proximal edge 122 of the second spacer layer 114 form portions 120, 122 of the side walls of the reaction chamber 124. The bottom layer 116 forms the bottom wall 126 of the reaction chamber 124. Antibodies 164 are tethered to the bottom 126 of the reaction chamber 124. The antigen-probe or pseudo-antigen-probe 162 is bound to the tethered antibodies 164.

A third shaped spacer layer 128, similar in shape to the first shaped spacer layer 112, is situated atop the first shaped spacer layer 112. A fourth spacer layer 130 has a slit 132 extending through the proximal end 134 of the spacer layer 130 towards the center of the spacer layer 130. The fourth spacer layer is 130 situated atop the second shaped spacer layer 114 with the proximal ends 122, 134 aligned. The slit 132 in the fourth spacer layer forms the sidewalls (not illustrated) of the detection chamber 132. The portion 138 of the second spacer layer exposed by the slit 132 in the fourth spacer layer 130 forms the bottom 138 of the detection chamber 132. The proximal end 140 of the slit 132 forms the passageway 140 between the reaction chamber 124 and the detection chamber 132. The proximal end 134 of the fourth spacer layer 130 forms a portion 134 of the sidewall of the reaction chamber 124.

A fifth shaped spacer 142, similar in shape to the first shaped spacer layer 112 and third shaped spacer layer 128, is situated atop the third spacer layer 128. A sixth shaped spacer layer 144, similar in shape to the second shaped spacer layer 114, is placed atop the fourth shaped spacer layer 130, with the proximal ends 146, 122 aligned. The portion 170 of the sixth spacer layer exposed by the slit 132 in the fourth spacer layer 130 forms the top 170 of the detection chamber 132. An aperture 148 extends through the sixth shaped spacer layer 144. The distal end 150 of the aperture 148 and the distal end 152 of the slit 132 are aligned. The aperture 148 forms a portion 150 of a sidewall of a vent 154, allowing displacement of air from the detection chamber 132 as it fills with sample. A top layer 156 is fitted over the fifth spacer layer 142 and sixth spacer layer 144. The top layer 156 also includes an aperture 158 of similar size and shape and in alignment with the aperture 148 in the sixth shaped layer 144.

In certain embodiments, it may be preferred to delay the filling of the detection chamber 132 to some time after sample has filled the reaction chamber 124, to allow time for the immuno-reactions to proceed in the reaction chamber 124. In these embodiments, this is achieved by forming a vent hole 158 in layer 116 and/or 156 after completion of the immuno-reactions. When the reaction chamber 124 fills with sample, air is trapped in the detection chamber 132, which prevents it from being filled with sample. At a suitable time after sample has filled the reaction chamber 124, at least one of the top layer 156 and the bottom layer 116 can be punctured above the vent hole 148 or below the vent hole 154 by a suitable device, such as a needle or blade. When this occurs, the air in the detection chamber 132 can vent through the hole 148 or hole 154 formed in layer 116 and/or 156 via aperture 148 or 154, thus allowing sample to be drawn into the detection chamber 132 from the reaction chamber 124 by capillary action and the displaced air to be vented.

The height of the detection chamber 132 is typically selected to be less than the height of the reaction chamber 124 such that, in combination with the surface energies of the faces of chambers 132 and 124, the capillary force in the detection chamber 132 will be greater than that in the reaction chamber 124. The stronger capillary force in the detection chamber 132 serves to draw sample into the detection chamber 132 while emptying the reaction chamber 124. This method of using differentials in capillary force to fill a chamber is described in detail in copending application Ser. No. 09/536,234 filed on Mar. 27, 2000.

In preferred embodiments, the height of the reaction chamber is typically greater than the height of the detection chamber. The height of the detection chamber is typically about 500 microns or less, preferably about 450, 400, 350, 300, 250 microns or less, and more preferably about 5, 10, 15, 20, 25, 30, 35, 40, 45, or 50 microns to about 75, 100, 125, 150, 175, or 200 microns. These detection chamber heights are particularly well suited to applications wherein the top and bottom walls of the detection chamber comprise electrode layers. However, there may be certain embodiments wherein electrochemical detection is employed wherein cell heights greater than about 500 microns may be preferred. These detection chamber heights may also be suitable when detection methods other than electrochemical detection are employed. When another detection method is employed, for example, an optical detection method, different cell heights may be preferred. In such embodiments, a cell height of about 600, 700, 800, or 900 microns or more, or even about 1, 1.5, 2, 2.5, 3, 3.5, 4, 4.5, or 5 mm or more may be preferred. The height of the reaction chamber is typically greater than that of the detection chamber. However, in certain embodiments it may be preferred to employ a reaction chamber having the same or a similar height as the detection chamber, or even a smaller height than the detection chamber. The detection chamber height is typically from about 5 microns or less to about 1, 1.5, 2, 2.5, 3, 3.5, 4, 4.5, or 5 mm or more, preferably about 900, 800, 700, 600, or 500 microns or less, more preferably about 450, 400, 350, 300, or 250 microns or less, and most preferably from about 5, 10, 15, 20, 25, 30, 35, 40, 45, or 50 microns to about 75, 100, 125, 150, 175, 200, or 250 microns.

When the immunosensor 100 is an electrochemical sensor 100, the top surface of the second spacer layer 138 and the bottom surface 160 of the sixth spacer layer 144 which are exposed by the slit 132 in the fourth spacer layer 130 may be partially or completely coated with a conducting material. Alternatively, layers 114 and 144 may themselves be made of electrically conductive materials. Electrical connection between the two conducting layers (not illustrated) and a meter (not illustrated) enable electrochemical measurements to be conducted within the detection chamber.

Fabrication Methods

For purposes of illustration, details of the fabrication of sensors of preferred embodiments are discussed with reference to the sensor depicted in FIGS. 3 and 4. The sensor strip 100 is typically constructed of layers of material laminated together. One or more spacer layers 128, 130 are used to space layers 112 and 114 apart from layers 142 and 144. The spacer layers have adhesive faces to allow layers 112, 128, and 142 and layers 114, 130, and 144 to be held together. Alternatively, the spacer layers themselves may consist of an adhesive, or may comprise a material capable of adhering to adjacent layers by the application of heat and/or pressure in a lamination method.

The detection chamber 132 is a capillary space where layers 114 and 144 form the end walls of the space and the thickness of layers 128, 130 define the height. Layers 114 and 144 can also serve as substrates for electrode coatings (not illustrated) that form the electrodes of an electrochemical cell or may act as the electrodes themselves by virtue of being constructed of electrically conductive materials. In construction, detection chamber 132 is typically formed by punching out, or otherwise removing a portion of layer 130. This cutout portion of layer 130 can also serve to define the electrode area of the electrochemical cell.

The reaction chamber 124 can be formed by punching or otherwise removing a portion of the spacer layers, with the areas removed such that the reaction chamber overlaps with the detection chamber 132, thus causing the detection chamber 132 to open into the reaction chamber 124. Layers 116 and 156 can then be laminated to the external face of layers 112, 114 and layers 142, 144, respectively, to form the end walls 126, 174 of reaction chamber 124. The immuno-chemicals 164 and 162 can be coated onto an internal face 126 and/or 174 of layers 116 and/or 156 prior to or following the lamination of 116 and 156 onto layers 112, 114 and layers 142, 144, respectively. Layers 116 and 156 can be adhered to layers 112, 114 and layers 142, 144, respectively, by an adhesive layer on the external face of layers 112, 114 and layers 142, 144, respectively, or on the internal face of layers 116 and 156.

The vent 148 and/or 154 can advantageously be formed by punching a hole through layers 114, 130, and 144. From the point of view of simplifying the strip fabrication process, it is particularly advantageous to form vent 148 and/or 154 at the same time as the cut-out portion for the reaction chamber 124 and/or the detection chamber 132 is formed, as this makes it easier to achieve a reproducible spatial relationship between the chamber(s) and the vent, and also reduces the number of process steps.

In a different embodiment, the vent 148, 154, 158, can be formed by punching through layers 114, 116, 130, 144, and 156 and additional tape layers (not illustrated) laminated over both ends of the hole thus formed. This has the advantage of permitting optimization of the properties of layers 116 and 156 and the vent hole covering tape layers (not illustrated) separately. Alternatively, vent hole 148, 154, 158 can be formed by punching through layers 114, 130, 144 and 116 or 156 prior to the lamination of layers 116 or 156, respectively. This leaves an opening of 158 to only one face of the strip 100 and thus only one covering tape is used.

In a further embodiment, the layers 116 and 156 can be formed and laminated to layers 114 and 144 such that layers 116 and 156 do not extend to cover the area where the vent 158 is formed. Then it is only necessary to punch through layers 114, 130 and 144 to form the vent 148, 154, 158 and additional tape layers (not illustrated) laminated over both ends of the hole thus formed.

The layers may be adhered to each other by any suitable method, for example, pressure sensitive adhesive, curable adhesives, hot melt adhesives, lamination by application of heat and/or pressure, mechanical fasteners, and the like.

The above-described configurations for the sensor are but two of many possible configurations for the sensor, as will be appreciated by one of skill in the art. For example, the vent may be provided through the top of the strip, the bottom of the strip, both the top and bottom of the strip, or through one or more sides of the strip. The vent may be of any suitable configuration, and may extend directly into a portion of the detection chamber, or may follow a circuitous path into the detection chamber. The detection chamber may be of any suitable shape, for example, rectangular, square, circular, or irregular. The detection chamber may abut the reaction chamber, or a separate sample passageway between the reaction chamber and the detection chamber may be provided. Sample may be admitted to the reaction chamber on either side of the strip, as in the sensor of FIGS. 4 and 3, or only through one side of the strip with the opposite side blocked by a spacer, as in FIGS. 1 and 2. The detection chamber may be of any suitable shape, for example, rectangular, square, circular, or irregular. The detection chamber may be contained within the body of the strip, and access to the detection chamber may be provided by one or more sample ingresses through the top, bottom, or sides of the strip. Typically, a particular configuration is selected such that the fabrication method may be simplified, e.g., by performing fewer steps or by using fewer components.

Electrochemical Detection

When the sensor is an electrochemical cell, the electrode layers, for example, layers 52 and 54 of the sensor of FIGS. 1 and 2, are provided with an electrical connector allowing the sensor 20 to be placed in a measuring circuit. At least one of the electrodes 52 or 54 in the cell 28 is a sensing electrode, i.e., an electrode sensitive to the amount of oxidized or reduced form of an analyte in the sample. In the case of a potentiometric sensor 20 wherein the potential of the sensing electrode 52 or 54 is indicative of the level of analyte present, a second electrode 54 or 52, acting as reference electrode is present which acts to provide a reference potential. In the case of an amperometric sensor 20 wherein the sensing electrode current is indicative of the level of analyte in the sample, at least one other electrode 54 or 52 is present which functions as a counter electrode to complete the electrical circuit. This second electrode 54 or 52 may also function as a reference electrode. Alternatively, a separate electrode (not shown) may perform the function of a reference electrode.

If the immunosensor 20 is operated as an electrochemical cell 28, then the sheet 36 containing the apertures defining the reaction chamber 22 and/or detection chamber 28 comprises an electrically resistive material. In a preferred embodiment, sheets 32 and 34 also comprise an electrically resistive material. Suitable electrically resistive materials include, for example, polyesters, polystyrenes, polycarbonates, polyolefins, mixtures thereof, and the like. Preferred polyester is polyethylene terephthalate. In the sensor depicted in FIGS. 1 and 2, the layers 32 and 34 are substrates coated with electrically conductive material 52 and 54. The electrically conductive material 52 or 54 is coated on the surface 60 or 62 facing the detection chamber 28 and an adhesive layer (not shown) is coated on the surface 33 or 35 facing layer 42 or 46, respectively.

In the embodiment depicted in FIGS. 3 and 4, the detection chamber 132 has electrically conductive coatings (not illustrated) on the internal face of 138 and 170 which are suitable for use as electrodes in an electrochemical sensor cell. Also contained in the detection chamber 132 is a dry reagent layer 172 comprising a substrate for the probe enzyme and, if necessary, a redox species capable of cycling the enzyme between its oxidized and reduced forms and capable of being oxidized or reduced at the cell electrodes. A buffer may also be present to control pH in the detection chamber 132. When the immunosensor is in use, the electrodes are connected to an external electronic meter device (not illustrated) through external connectors (not illustrated), for example, tongue plugs, as are known in the art. Suitable connectors are disclosed in copending application Ser. No. 09/399,512 filed on Sep. 20, 1999 and copending Application No. 60/345,743 filed on Jan. 4, 2002.

If the immunosensor 20, 100 is operated using a detection method other than an electrochemical detection method, then the materials from which the sensor is constructed need not be electrically resistive. However, the polymeric materials described above are preferred for use in constructing the immunosensors of preferred embodiments because of their ease of processing, low cost, and lack of reactivity to reagents and samples.

Optical Detection

In an alternative embodiment, an optical rather than an electrochemical detection system are used. According to this alternative embodiment, electrodes are not necessary and an external light source and photocell are used to analyze light transmitted through, or reflected from the solution in detection chamber. In one embodiment, it is preferred to shine the light through the top surface of the sensor then through the sample, where it is reflected off the lower sensor layer and then back up through the sample and the top layer, where it is detected. In another embodiment, light is shone in through the side of the detection chamber and totally internally reflected between the end faces of the detection chamber until it passes out through the other side of the detection chamber, where it is detected. In these embodiments, the layers above, to the side, and/or below the detection chamber are substantially transparent to the analyzing light that is passed through the layer or layers. The techniques described in copending application Ser. No. 09/404,119 filed on Sep. 23, 1999 may be adapted for use with the immunosensors of preferred embodiments utilizing optical detection systems. Alternatively, in certain embodiments it may be preferred to use a combination of electrochemical detection and optical detection methods, which is also described in application Ser. No. 09/404,119.

Reagents and Other Materials Present in the Immunosensor

Reagents for use in the reaction chamber, e.g., immobilized antibody, pseudo-antigen-probe, buffer, mediator, and the like, may be supported on the walls of the reaction chamber or on the walls of the detection chamber, on an independent support contained within chambers, within a matrix, or may be self supporting. If the reagents are to be supported on the chamber walls or electrodes, the chemicals may be applied by use of printing techniques well known in the art, e.g., ink jet printing, screen printing, slot coating, lithography, and the like. In a preferred embodiment, a solution containing the reagent is applied to a surface within a chamber and allowed to dry.

Rather than immobilize or dry the reagents or other chemicals onto the surfaces of the reaction chamber or detection chamber, it may be advantageous to support them on or contain them within one or more independent supports, which are then placed into a chamber. Suitable independent supports include, but are not limited to, mesh materials, nonwoven sheet materials, fibrous filling materials, macroporous membranes, sintered powders, gels, or beads. The advantages of independent supports include an increased surface area, thus allowing more antibody and pseudo-antigen-probe to be included in the reaction chamber, if desired. In such an embodiment, the antibody bound to the pseudo-antigen-probe is dried onto a piece of porous material, which is then placed in the reaction chamber. It is also easier during fabrication to wash unbound protein from independent supports, such as beads, compared to washing unbound protein off of the surface of the reaction chamber.

In a particularly preferred embodiment, the antibody bound to the pseudo-antigen-probe is supported on beads. Such beads may comprise a polymeric material, e.g., latex or agarose, optionally encasing a magnetic material (such as gamma Fe₂O₃ and Fe₃O₄). The bead material is selected such that suitable support for the antibody is provided. Suitable beads may include those marketed as DYNABEADS® by Dynal Biotech of Oslo, Norway. Optionally, a magnet may be included in the meter to hold the magnetic beads in the reaction chamber and to stop them from moving to the detection chamber.

In yet another embodiment, the walls of the reaction chamber are porous, with the antibody bound to the pseudo-antigen-probe incorporated into the pores. In this embodiment, the liquid sample is able to wick into the porous wall, but not leak out of the defined area. This is accomplished by using a macroporous membrane to form the reaction chamber wall and compressing the membrane around the reaction chamber to prevent leakage of sample out of the desired area, as described in U.S. Pat. No. 5,980,709 to Hodges, et al.

Suitable independent supports such as beads, mesh materials, nonwoven sheet materials, and fibrous fill materials include, polyolefins, polyesters, nylons, cellulose, polystyrenes, polycarbonates, polysulfones, mixtures thereof, and the like. Suitable macroporous membranes may be prepared from polymeric materials including polysulfones, polyvinylidene difluorides, nylons, cellulose acetates, polymethacrylates, polyacrylates, mixtures thereof, and the like.

The antibody bound to the pseudo-antigen-probe may be contained within a matrix, e.g., polyvinyl acetate. By varying the solubility characteristics of the matrix in the sample, controlled release of the protein or antibody into the sample may be achieved.

As illustrated in FIG. 2, dried reagents 64 may optionally be disposed in the detection chamber 28. These reagents may include an enzyme substrate (used as a probe) and a mediator, capable of reacting with the enzyme part of the pseudo-antigen-enzyme probe 50 to produce a detectable signal. The enzyme substrate and mediator, if present, are to be of sufficient amount such that the rate of reaction of any enzyme present with the enzyme substrate 64 is determined by the amount of enzyme present. For instance, if the enzyme is glucose oxidase or glucose dehydrogenase, a suitable enzyme mediator 64 and glucose (if not already present in the sample) is disposed into the detection chamber 28.

In an embodiment wherein an electrochemical detection system is used, ferricyanide is a suitable mediator. Other suitable mediators include dichlorophenolindophenol and complexes between transition metals and nitrogen-containing heteroatomic species. Buffer may also be included to adjust the pH of the sample in the detection chamber 28, if necessary. The glucose, mediator, and buffer reagents 64 are present in sufficient quantities such that the rate of reaction of the enzyme with the enzyme substrate 64 is limited by the concentration of the enzyme present.

The internal surface 40 of the substrate 42, which forms the base of the reaction chamber 22, is coated with pseudo-antigen-probe 50 bound to antibodies 44 to the antigen to be detected in the sample. The antibodies 44 are adsorbed or otherwise immobilized on the surface 40 of the substrate 42 such that they are not removed from the substrate 42 during a test. Optionally, during or after application of the antibodies 44 to the internal surface 40 of the substrate 42, an agent designed to prevent non-specific binding of proteins to this surface can be applied (not shown). An example of such an agent well known in the art is bovine serum albumin (BSA). A nonionic surfactant may also be used as such an agent, e.g., TRITON® ×100 surfactant manufactured by Rohm & Haas of Philadelphia, Pa., or TWEEN® surfactants manufactured by ICI Americas of Wilmington, Del. The nonionic surfactant selected does not denature proteins. The coating 44 on the internal surface 40 of the substrate 42 is in the dry state when ready to be used in a test.

In preferred embodiments wherein electrochemical detection is employed, enzymes may be used as the probe. Examples of suitable enzymes include, but are not limited to, horseradish peroxidase, glucose oxidase, and glucose dehydrogenase, for example, PQQ dependent glucose dehydrogenase or NAD dependent glucose dehydrogenase.

The probe can also be an enzyme co-factor. Examples of suitable cofactors include, but are not limited to, flavin mononucleotide, flavin adenine dinucleotide, nicotinamide adenine dinucleotide, and pyrroloquinoline quinone. The co-factor is preferably linked to the antigen by a flexible spacer to allow the co-factor to bind to the apoenzyme. When the probe is a co-factor, the apoenzyme may optionally be co-dried with the enzyme substrate and mediator in the reaction chamber.

The probe can also be a regulator of enzyme activity. Examples of suitable enzyme regulators include, but are not limited to, kinases or phosphorylases. Enzyme regulators may alter the activity of the enzyme by changing the state of phosphorylation, methylation, adenylation, uridylation or adenosine diphosphate ribosylation of the enzyme. Enzyme regulators may also alter the activity of the enzyme by cleaving a peptide off the enzyme. When the probe is an enzyme regulator, the enzyme is co-dried with the enzyme substrate and mediator in the reaction chamber.

The probe can be a protein subunit which is part of a multi-subunit complex. An example of such a protein subunit is one of the subunits in the multi-subunit enzyme cytochrome oxidase.

The antibody and pseudo-antigen-probe can be complexed together before being dried into the reaction chamber. Complexation conditions are chosen to minimize the amount of free (uncomplexed) pseudo-antigen-probe, as this species will increase the background signal in the assay. The amount of free antibody is also minimized as this species will bind antigen and stop it from displacing the pseudo-antigen-probe, thus reducing the sensitivity of the assay. For example, it is possible to optimize the complexation of pseudo-antigen-probes with antibodies by “crowding” the solutions with inert macromolecules, such as polyethylene glycol, which excludes volume to the proteins and thus raises their thermodynamic activity and enhances the affinity of their binding to one another. See, e.g., Minton, Biopolymers, Vol. 20, pp 2093-2120 (1981).

It is advantageous to have the antibody immobilized on beads before it is complexed to the pseudo-antigen-probe. This allows all the antibody sites to be occupied by exposing them to a high concentration of the pseudo-antigen-probe. Excess pseudo-antigen-probe is then readily removed by centrifugation and washing of the beads.

The immunosensor is most sensitive to antigen concentrations from about 1 nM to about 10 μM (micromolar). For an antigen with a relative molar mass of 100,000, this corresponds to about 0.1 μg/mL (micrograms/mL) to about 1000 μg/mL (micrograms/mL). However, the sensor can be modified (e.g., by changing the separation between the electrodes, or by applying a different pattern of voltage pulses) to assay antigen concentrations in the range 0.1 nM or less to 0.1 mM or more.

The maximum detectable limit of the assay is determined by the concentration of pseudo-antigen-probe/antibody in the reaction chamber. This molar concentration is therefore set to correspond to the expected range of molar antigen concentrations that are typically encountered in samples of interest. For example, the concentration of C-reactive protein encountered in a typical pathology laboratory is from about 10 nM to about 10 μM (micromolar).

Examples of antigens that may be assayed include, but are not limited to, Alpha-fetoprotein, Carcinoembryonic antigen, C-reactive protein, cardiac Troponin I, cardiac Troponin T, Digoxin, ferritin, Gamma glutamyl transferase, Glycated hemoglobin, glycated protein, Hepatitis A, B and C, chorionic gonadotropin, Human immunodeficiency virus, insulin, serum amyloid A, thromblastin, Prostate specific antigen, Prothrombin, Thyroxine, Tumor antigen CA125, Tumor antigen CA15-3, Tumor antigen CA27/29, Tumor antigen CA19-9, and Tumor antigen NMP22.

The sensors of preferred embodiments are not limited to the assay of human antigens, but are also suitable for use in veterinary and animal husbandry applications. Also, if an antigen is too small to be immunogenic, then it can be attached to a carrier as a hapten and antibodies can be raised to it in this way. Therefore the invention is not limited to the assay of protein antigens or to large molecules, but is also applicable to small antigens as well.

Antibodies suitable for use in the sensors of preferred embodiments include, but are not limited to, the natural antibodies, such as IgG, IgM and IgA. Suitable antibodies can also be made up of fragments of natural antibodies, such as F(ab)₂ or Fab. The antibody can be composed of genetically engineered or synthetic fragments of natural antibodies, such as scFv (single chain Fragment variable) species.

The antibodies can be complexed to native antigen probes or to “pseudoantigen” probes. Examples of pseudo-antigens include antigens from other species. For example, if human C-reactive protein is to be assayed then the pseudo-antigen may include canine, feline, equine, bovine, ovine, porcine or avian C-reactive protein. Pseudo-antigens can also be made by modifying the native antigen. For example, if human C-reactive protein is to be assayed, then the pseudo-antigen may include a monomeric form of the native pentamer, or C-reactive protein which has had its amine, carboxyl, hydroxyl, thiol or disulfide groups chemically modified.

Using the Sensor to Determine the Presence or Absence of an Antigen

The sensor may be used to determine the presence or absence of an antigen in a sample as follows. Referring to FIGS. 3 and 4, the strip sensor 100 contains a reaction chamber 124 and a detection chamber 132. Sample is introduced into reaction chamber 124 via port 166 or 168. The separation between layers 116 and 156 and the surface energy of their internal surfaces is such that the sample will be drawn into reaction chamber 124 by capillary action. Reaction chamber 124 contains antibodies 164 immobilized to an internal face 126 of the reaction chamber 124. Pseudo-antigen-probe complexes 162 are bound to antibodies 164 such that substantially all the antibody recognition sites for the antigen are blocked by pseudo-antigen-probe 162. In this embodiment, the probe is an enzyme.

In FIG. 4, the antibody is shown as coated only on one face 126 of the reaction chamber 124, but it may advantageously be coated on more than one face 126 of the reaction chamber 124 or coated onto a separate support (not illustrated) that is contained in the reaction chamber 124. However, for ease of fabrication it is typically preferred that the antibodies 164 are only coated on one portion of the reaction chamber 124, or on a single support material. When a separate support is used to immobilize the antibodies 164, the support is such that it does not enter the detection chamber 132 during the test. This can be achieved by, for example, adhering the support to at least one face 126 of the reaction chamber 124, or by selecting the size or shape of the support such that it cannot enter through the sample passageway 134 into detection chamber 132, or by selecting a support of sufficient density such that it remains on the lower face 126 of the reaction chamber 124 when the sample is transferred to the detection chamber 132.

When sample fills the reaction chamber 124, the pseudo-antigen-enzyme probe 162 bound to antibody 164 contacts the sample and a small fraction of the pseudo-antigen-probe dissociates from the antibody 164 and into the sample. Sufficient time is then allowed for the dynamic equilibrium between bound and unbound pseudo-antigen-enzyme probe 162 to be established. If antigen is present in the sample, the antigen, which binds more strongly to the antibody 164 than the pseudo-antigen-enzyme probe 162, eventually displaces the pseudo-antigen-enzyme probe 162. Thus each antigen that binds to an immobilized antibody 164 will displace one pseudo-antigen-enzyme probe 162 into solution.

The end of the reaction step is a predetermined time after the sample is introduced into the reaction chamber 124. The predetermined time is set such that there is sufficient time for substantially all of the antigen in the sample to displace pseudo-antigen-enzyme probe 162 to bind to the antibody 164. Alternatively, the predetermined time can be set such that a known fraction of the antigen displaces the pseudo-antigen-probe 162 to bind to the antibody 164.

The time that the sample is introduced into the reaction chamber 124 can be indicated by the user, for example, by depressing a button on a meter (not illustrated) connected to the sensor 100. This action is used to trigger a timing device (not illustrated). In the case of visual detection, no meter device is necessary. In such an embodiment, the user manually times the reaction period.

In the case where electrochemical detection is used to detect the result of the immuno-reactions, the indication that sample has been introduced into the reaction chamber 124 can be automated. As described above, when sample fills the reaction chamber 124, a small portion of the detection chamber 132 at its opening 140 into the reaction chamber 124 will be wet by sample. If electrochemical detection is employed then at least two electrodes (not illustrated) are present in the detection chamber 132. If these electrodes (not illustrated) are placed in the detection chamber 134, such that at least a portion of each electrode (not illustrated) is contacted by the sample during the filling of the reaction chamber 124, the presence of the sample will bridge the electrodes (not illustrated) and create an electrical signal which can be used to trigger the timing device.

A predetermined time after the timing device has been triggered, either by the user or automatically, the immuno-reaction phase of the test is deemed to be completed. When the immuno-reaction phase of the test is completed, the vent 158 to the atmosphere is opened. For example, a solenoid activated needle in the meter may be used to pierce layer 156 and/or layer 116, or additionally layers 114 and 44, thus opening the distal end 152 of the detection chamber 132 to the atmosphere. The piercing can be automatically performed by the meter, as in the example above, or manually by the user in the case of visual detection wherein no meter may be used, e.g., the user inserts a needle through the layers 156, 116, 114, and/or 144 into the detection chamber, thereby forming the vent 158.

The opening of the vent 158 to the atmosphere allows the air trapped in the detection chamber 132 to escape, thereby allowing the detection chamber 132 to be filled with reacted sample from the reaction chamber 124. The reacted sample will be drawn into the detection chamber 132 due to increased capillary force in the detection chamber 132 compared to that present in the reaction chamber 124. In a preferred embodiment, the increased capillary force is provided by suitably coating the surfaces 138 and 160 of the detection chamber 132 or, more preferably, by choosing the capillary distance for the detection chamber 132 to be smaller than that of the reaction chamber 124. In this embodiment, the capillary distance is defined to be the smallest dimension of the chamber.

When the detection chamber 132 is filled, the reagents 172 dissolve into the sample. The enzyme component of the reagent layer 172 reacts with the enzyme substrate and the mediator to produce reduced mediator. This reduced mediator is electrochemically oxidized at an electrode (not illustrated) acting as an anode in the detection chamber 134 to produce an electrical current. In one embodiment, the rate of change of this current with time is used as an indicator of the presence and amount of enzyme that is present in the reacted sample. If the rate of change of current is less than a predetermined threshold value (taking into account that some pseudo-antigen-enzyme probe 162 is liberated into solution as a result of the dynamic equilibrium that is established between the free and bound pseudo-antigen-enzyme probe 162), then it is indicative of no significant amount of pseudo-antigen-enzyme probe 162 present in the reacted sample, indicating the lack of antigen present in the original sample. If the rate of change of current is higher than the threshold rate, it indicates that pseudo-antigen-enzyme probe 162 is present in the reacted sample in an amount greater than the threshold value, and thus antigen is also present in the sample initially. In one embodiment, the rate of change of the current is used to give a measure of the relative amount of antigen initially present in the sample.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention as embodied in the attached claims. All patents, applications, and other references cited herein are hereby incorporated by reference in their entirety.

What is claimed is:

1. A disposable device for use in detecting a target antigen in a fluid sample, the device comprising a reaction chamber; an immobilized antibody fixed within the reaction chamber; a reporter complex comprising a probe and a reporter complex antigen, wherein the probe is linked to the reporter complex antigen, wherein the reporter complex antigen is bound to the immobilized antibody, and wherein the reporter complex antigen binds less strongly than the target antigen to the immobilized antibody; a detection chamber; a sample ingress to the reaction chamber; and a sample passageway between the reaction chamber and the detection chamber.

2. The device of claim 1, wherein the reporter complex antigen is selected from the group consisting of the target antigen, a pseudo-antigen, and a modified-antigen.

3. The device of claim 1, wherein the probe is selected from the group consisting of radioisotopes, chromophores, and fluorophores.

4. The device of claim 1, wherein the probe comprises an enzyme.

5. The device of claim 4, wherein the enzyme comprises a glucose dehydrogenase.

6. The device of claim 4, further comprising an enzyme substrate.

7. The device of claim 6, wherein the enzyme substrate is an oxidizable substrate.

8. The device of claim 7, wherein the enzyme substrate comprises glucose.

9. The device of claim 6, further comprising a mediator.

10. The device of claim 9, wherein the mediator is selected from the group consisting of dichlorophenolindophenol and complexes between transition metals and nitrogen-containing heteroatomic species.

11. The device of claim 10, wherein the mediator comprises ferricyanide.

12. The device of claim 4, wherein the sample has a pH, and wherein the device further comprises a buffer that adjusts the pH of the sample.

13. The device of claim 12, wherein the buffer comprises a phosphate.

14. The device of claim 12, wherein the buffer comprises a mellitate.

15. The device of claim 4, further comprising a stabilizer, wherein the stabilizer stabilizes at least one component selected from the group consisting of the target antigen, the reporter complex antigen, the enzyme, and the immobilized antibody.

16. The device of claim 6, wherein the enzyme substrate is supported on a detection chamber interior surface.

17. The device of claim 1, wherein the immobilized antibody is supported on a reaction chamber interior surface.

18. The device of claim 1, further comprising a support material.

19. The device of claim 18, wherein the support material is contained within the detection chamber, and wherein a first substance selected from the group consisting of an enzyme substrate, a mediator, and a buffer is supported on or contained within the support material.

20. The device of claim 18, wherein the support material is contained within the reaction chamber, and wherein a second substance selected from the group consisting of the immobilized antibody, the reporter complex, and an agent that prevents non-specific binding of proteins to a reaction chamber internal surface is supported on or contained within the support material.

21. The device of claim 18, wherein the support material comprises a mesh material.

22. The device of claim 21, wherein the mesh material comprises a polymer selected from the group consisting of polyolefin, polyester, nylon, cellulose, polystyrene, polycarbonate, polysulfone, and mixtures thereof.

23. The device of claim 18, wherein the support material comprises a fibrous filling material.

24. The device of claim 23, wherein the fibrous filling material comprises a polymer selected from the group consisting of polyolefin, polyester, nylon, cellulose, polystyrene, polycarbonate, polysulfone, and mixtures thereof.

25. The device of claim 18, wherein the support material comprises a porous material.

26. The device of claim 25, wherein the porous material comprises a sintered powder.

27. The device of claim 25, wherein the porous material comprises a macroporous membrane.

28. The device of claim 27, wherein the macroporous membrane comprises a polymeric material selected from the group consisting of polysulfone, polyvinylidene difluoride, nylon, cellulose acetate, polymethacrylate, polyacrylate, and mixtures thereof.

29. The device of claim 18, wherein the support material comprises a bead.

30. The device of claim 1, wherein the detection chamber comprises a first electrode and a second electrode.

31. The device of claim 30, wherein at least one of the first electrode and the second electrode comprises a material selected from the group consisting of aluminum, copper, nickel, chromium, steel, stainless steel, palladium, platinum, gold, iridium, carbon, carbon mixed with binder, indium oxide, tin oxide, a conducting polymer, and mixtures thereof.

32. The device of claim 1, wherein a detection chamber wall is transparent to a radiation emitted or absorbed by the probe, wherein the radiation is indicative of a presence or absence of the reporter complex in the detection chamber.

33. The device of claim 1, further comprising a detector that detects a condition wherein the reaction chamber is substantially filled.

34. The device of claim 1, further comprising a piercing means that forms a detection chamber vent in a distal end of the detection chamber.

35. The device of claim 1, further comprising a reaction chamber vent at a distal end of the reaction chamber.

36. The device of claim 1, wherein the target antigen comprises a human C-reactive protein.

37. The device of claim 36, wherein the reporter complex antigen comprises a monomeric C-reactive protein.

38. The device of claim 36, wherein the reporter complex antigen comprises a C-reactive protein derived from a non-human species.

39. The device of claim 36, wherein the reporter complex antigen comprises a chemically-modified C-reactive protein, wherein an affinity of the chemically-modified C-reactive protein to the antibody is less than an affinity of the human C-reactive protein to the antibody

40. The device of claim 1, wherein a wall of the detection chamber or a wall of the reaction chamber comprises a material selected from the group consisting of polyester, polystyrene, polycarbonate, polyolefin, polyethylene terephthalate, and mixtures thereof.

41. The device of claim 40, wherein the wall of the detection chamber or the wall of the reaction chamber further comprises a filler.

42. The device of claim 41, wherein the filler is a filler material selected from the group consisting of titanium dioxide, carbon, silica, glass, and mixtures thereof.

43. The device of claim 1, wherein the probe comprises an enzyme co-factor.

44. The device of claim 43, wherein the enzyme co-factor is selected from the group consisting of flavin mononucleotide, flavin adenine dinucleotide, nicotinamide adenine dinucleotide, and pyrroloquinoline quinone.

45. The device of claim 43, wherein the enzyme co-factor is linked to the reporter complex antigen through a flexible spacer.

46. The device of claim 43, further comprising an enzyme substrate.

47. The device of claim 43, further comprising an apoenzyme.

48. The device of claim 1, wherein the probe comprises an enzyme activity regulator.

49. The device of claim 48, wherein the enzyme activity regulator comprises a kinase or phosphorylase.

50. The device of claim 48, further comprising an enzyme substrate.

51. The device of claim 48, further comprising an enzyme.

52. The device of claim 1, wherein the probe comprises a protein subunit of a multi-subunit enzyme.

53. A method for determining an amount of a target antigen in a fluid sample, the method comprising the steps of:
placing the fluid sample in a reaction chamber containing an immobilized antibody and a reporter complex comprising a probe linked to a reporter complex antigen, wherein the antibody is fixed within the reaction chamber, wherein the reporter complex antigen is bound to the immobilized antibody, and wherein the reporter complex antigen binds less strongly than the target antigen to the immobilized antibody;
dissociating a portion of the reporter complex antigen from the immobilized antibody into the fluid sample;
binding a portion of the target antigen to the immobilized antibody;
transferring the fluid sample to a detection chamber; and
determining an amount of reporter complex in the fluid sample, wherein the amount of reporter complex is indicative of the amount of target antigen initially in the fluid sample.

54. The method of claim 53, wherein the step of transferring the fluid sample to a detection chamber comprises transferring the fluid sample to an electrochemical cell, the electrochemical cell comprising a first electrode and a second electrode.

55. The method of claim 54, wherein the step of determining an amount of reporter complex in the fluid sample comprises:
applying a potential between the first electrode and the second electrode; and
measuring a current, wherein the current is indicative of an amount of reporter complex present in the fluid sample, and wherein the amount of reporter complex is indicative of the amount of target antigen initially in the fluid sample.

56. The method of claim 53, wherein the step of transferring the fluid sample to a detection chamber comprises transferring the fluid sample to a detection chamber comprising an electromagnetic radiation transmissive portion.

57. The method of claim 56, wherein the step of determining an amount of reporter complex in the fluid sample comprises the steps of:
exposing the electromagnetic radiation transmissive portion to electromagnetic radiation, whereby the electromagnetic radiation passes through the fluid sample or reflects from the fluid sample; and
monitoring a property of the electromagnetic radiation after it passes through the fluid sample or reflects from the fluid sample, wherein the property is indicative of an amount of reporter complex present in the fluid sample, and wherein the amount of reporter complex is indicative of the amount of target antigen initially in the fluid sample.
